(19) United States
(12) Patent Application Publication
(10) Pub. No.: US 2001/0054425 A1
(43) Pub. Date: Dec. 27, 2001

(54) Hyperpharyngeal tube

(76) Inventor: Volker Bertram, Sulz/Neckar (DE)

Correspondence Address: Eugene LeDonne, Esq., Reed Smith LLP, 375 Park Ave, 17th Floor, New York, NY, 10152, US

(21) Appl. No.: 09/837,058

(22) Filed: Apr. 18, 2001

(30) Foreign Application Priority Data
Apr. 20, 2000 (DE) 100 19 956.9

(51) Int. Cl. A61M 15/00; A61M 16/10; A62B 9/06; A61M 16/00

(52) U.S. Cl. 128/207.15; 128/207.14; 128/203.12

(57) ABSTRACT

A hyperpharyngeal tube, in particular for intubation anesthesia, comprises a dimensionally stable tube shaft which is provided with an inflatable first cuff for sealing the oro- and nasopharynx and fixing the hyperpharyngeal tube in the pharynx, and an inflatable second cuff for sealing the esophagus. The lumen of the tube shaft is divided by a separating wall into a suction channel and a respiratory channel.

### RELATED APPLICATIONS

This application claims Paris Convention priority of German patent application number 100 19 956.9 filed on Apr. 20, 2000, the complete disclosure of which is hereby incorporated by reference.

### BACKGROUND OF THE INVENTION

A Field of the Invention

The invention concerns a hyperpharyngeal tube, in particular for intubation anesthesia, comprising a dimensionally stable tube shaft provided with an inflatable first cuff for sealing the naso- and oropharynx and fixing the hyperpharyngeal tube in the pharynx, and an inflatable second cuff for sealing the esophagus.

B Description of the Background of the Invention A comparable tube is disclosed e.g. in DE 195 37 735 C1. A tube of this type represents an alternative to a breathing mask, a so-called larynx mask, or to an endotracheal tube if there is no danger of aspiration. A tube of this type is an ideal supplement for emergency artificial respiration due to its simplicity.

In a possible application of intubation anesthesia, the tube is introduced into the patient, in most cases after premedication, after initial intra-venous anesthesia or after inhalation anesthesia. If the tube tip is introduced only to the opening region of the esophagus or into the hyperpharynx and the esophageal cuff blocks only this opening region, painful stress of the esophagus of the patient is prevented. The activated esophageal cuff occludes the entrance to the esophagus in a gas and liquid-tight fashion. When the tube has been introduced, blockage of the cuffs in the esophagus and pharynx produces a closed connection between the upper and lower respiratory tract, i.e. via the larynx into the trachea. Artificial respiration of the patient is now possible through the application channel in the inside of the hyperpharyngeal tube. Optionally, inhalation anesthesia may follow through supply of an anaesthetic, e.g. an air or O2 anaesthetic mixture. At the same time there is an open connection to the esophagus such that stomach vomit and pressures produced thereby can get from the stomach to the outside, i.e. stomach content and respiratory air cannot mix. Moreover, there is the possibility of permanent suction without impairing or disturbing artificial respiration through the application channel.

When the tube has been introduced, it may become necessary to remove secretion or foreign bodies from the esophagus.

It is the underlying purpose of the present invention to produce a tube of the above-mentioned type having an as simple as possible construction, which permits, in addition to artificial respiration also suction of secretions or foreign bodies from the esophagus and, at any time, release of stomach content and overpressures to the outside.

### SUMMARY OF THE INVENTION

This object is achieved in accordance with a hyperpharyngeal tube, in particular for emergency artificial respiration and intubation anesthesia with a dimensionally stable tube shaft whose lumen is subdivided by a separating wall into a suction channel and an artificial respiratory channel. The suction channel is thereby integrated in the tube such that the outer shape of the tube including suction channel is the same as of the known tube without suction channel.

In a further development of the invention, the tube shaft has such a length that the introduced tube shaft terminates in the hyperpharynx. The shaft end cannot accidentally be introduced into the trachea or the esophagus.

The suction channel preferably comprises in the region of the shaft end facing the esophagus, a first opening and a second opening in the region of the shaft end facing away from the esophagus. The suction channel is continuously open and offers a permanently available suction means.

One embodiment of the inventive transpharyngeal tube is shown in the schematic drawing and is further explained in the following description with reference to the drawing.

### BRIEF DESCRIPTION OF DRAWINGS

FIG. 1 shows the entrance region of the esophagus;

FIG. 2 shows a longitudinal section through a hyperpharyngeal tube;

FIG. 3 shows the lower side of the hyperpharyngeal tube according to FIG. 2; and

FIG. 4 shows a cross-section of the hyperpharyngeal tube along a line III-III according to FIG. 2.

### DETAILED DESCRIPTION OF A PREFERRED EMBODIMENT OF THE INVENTION

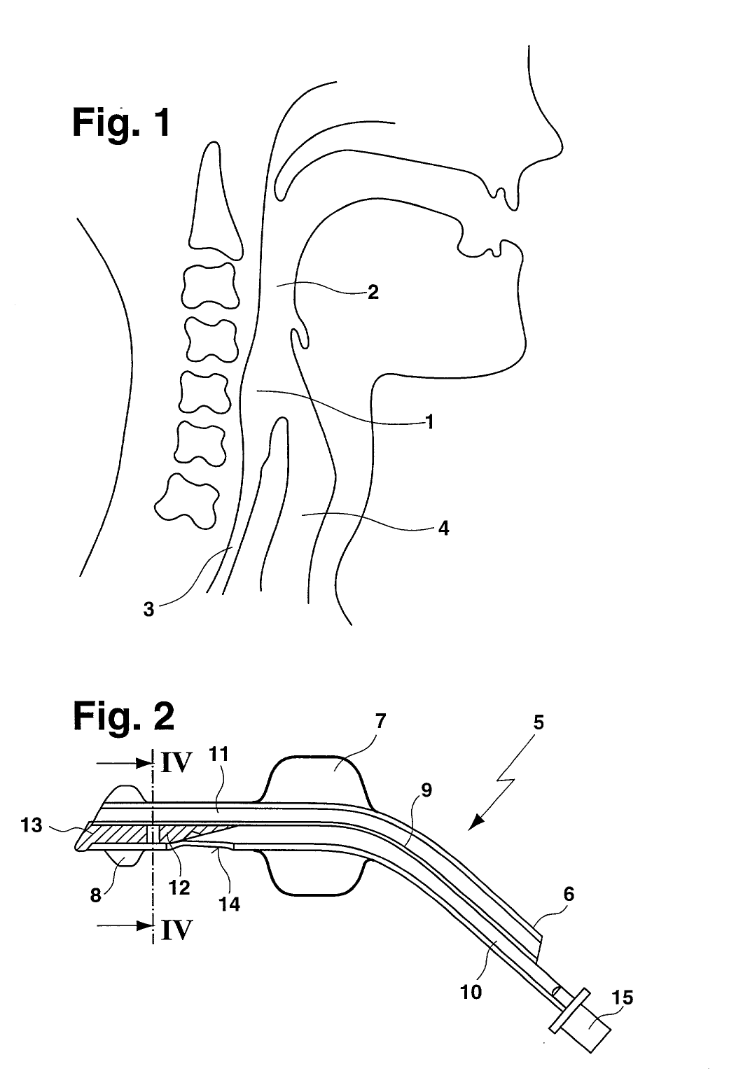

FIG. 1 shows the application region of a hyperpharyngeal tube shown in FIGS. 2 through 4, whose tube tip can be introduced into the hyperpharynx 1, the region between oro- or nasopharynx 2 and esophagus 3. Access to the esophagus 3 is blocked and sealed. The trachea 4 can be artificially respired by means of the hyperpharyngeal tube.

In accordance with FIG. 2, the hyperpharyngeal tube 5 comprises a substantially dimensionally stable tube shaft 6 of silicone or another plastic material which has a curved longitudinal profile and is straight at least in the region of two cuffs 7 and 8 or is S-shaped. The longitudinal profile of this design guarantees that the hyperpharyngeal tube 5 is always only inserted to the hyperpharynx (see FIG. 1). Due to the short length and shape of the hyperpharyngeal tube 5, insertion of the hyperpharyngeal tube 5 into the trachea is prevented. Both cuffs 7 and 8 are cylindrical. The cuff 8 abuts the hyperpharynx and blocks the esophagus. The cuff 7 is located in the oropharynx, blocks the oro- and nasopharynx and stabilizes the position of the hyperpharyngeal tube. Both cuffs 7 and 8 are simultaneously ventilated thereby assuring that the cuff 7 is ventilated first and subsequently cuff 8. This can be achieved by one single inflation line wherein the air supply can be influenced via a suitable free flow cross-sectional surface. Alternatively, ventilation of both cuffs 7 and 8 through two separate inflation lines is also possible. Both cuffs 7,8 have a cylindrical design. Cuffs of an embodiment (not shown) of the invention may have a symmetrical design or an anatomically formed shape.

The lumen of the hyperpharyngeal tube 5 is divided by a separating wall 9 into an artificial respiratory channel 10 and a suction channel 11. The suction channel 9 provides an open connection between mouth opening and esophagus and permits removal of secretions or foreign bodies from the esophagus. Both channels 10 and 11 terminate in the hyperpharynx when the hyperpharyngeal tube 5 is properly applied. A wedge 12 forms a flow aid to guide respiratory air directly into the trachea. The wedge 12 may also assist introduction of a suction catheter or a fiber-optic bronchoscope into the trachea. The respiratory channel 10 is provided with a 15 mm ISO standard connector 15 to prevent mixing up of respiration and suction. The suction channel 11 is always open in both directions to permit suction at any time.

Since the tube shaft 6 is formed of a flexible plastic material, the tube tip 13 inserted into a shaft end is slightly pressed to the inner wall of the hyperpharynx due to the spring effect of the tube shaft 6 thereby fixing the tube tip. To fix the tube tip 13 in this position, the cuff 8 is ventilated which blocks the entrance region of the esophagus in a gas and liquid-tight fashion. The inserted soft and flexible tube tip prevents injuries during insertion of the tube.

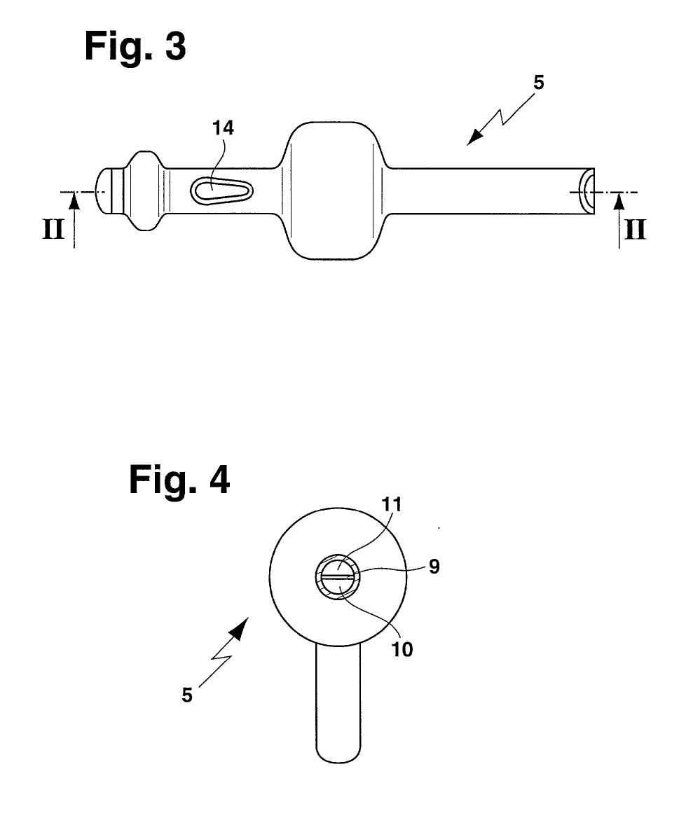

The respiratory channel 10 comprises a ventilation opening 14 via which the patient to be treated is provided with air and at the same time can be treated with application means, e.g. anesthetics. The application means can flow into the trachea. The ventilation opening 14 is better shown in FIG. 3.

Division of the lumen of the hyperpharyngeal tube 5 by the separating wall 9 into two channels 10 and 11 is more clearly shown in FIG. 4. For reasons of clarity, the wedge 12 is not shown in FIG. 4. The uniform continuous channel 10 always provides access to the esophagus independent of the application through the channel 11.

While the invention has been particularly shown and described with respect to illustrative and preferred embodiments thereof, it will be understood by those skilled in the art that the foregoing and other changes in form and details may be made therein without departing from the spirit and scope of the invention that should be limited only by the scope of the appended claims.

I claim:

1. Hyperpharyngeal tube for intubation anesthesia, said tube comprising:
a dimensionally stable tube shaft, which includes an inflatable first cuff for sealing the naso and oro-pharynx and fixing the hyperpharyngeal tube in the pharynx; and
an inflatable second cuff for sealing the oesophagus;
wherein a lumen of the tube shaft is divided by a separating wall into a suction channel for removing secretions or foreign bodies from the esophagus and into a respiratory channel comprising a ventilation opening for guiding respiratory air into a trachea.

2. The hyperpharyngeal tube according to claim 1, wherein the tube shaft has such a length that when introduced into a naso and oro-pharynx, said tube shaft terminates in a hyperpharynx.

3. The hyperpharyngeal tube according to claim 1, wherein the suction channel includes a first opening in the region of the shaft end of the tube shaft facing the esophagus and a second opening in the region of the shaft end facing away from the esophagus and the respiratory channel is always provided with a 15 mm ISO standard connector for respiration to prevent confusion

4. A tube shaft for use in hyperpharyngeal intubation, said tube shaft comprising:
a first cuff, said first cuff being used for sealing the naso and oro-pharynx and fixing the hyperpharyngeal tube in the pharynx,
a second cuff for sealing the oesophagus, and
a separating wall for separating a lumen of the tube shaft into a first channel and a second channel.

5. The tube shaft of claim 4, wherein said tube shaft is dimensionally stable.

6. The tube shaft of claim 5, wherein said first channel is used for removing secretions and foreign bodies from an esophagus.

7. The tube shaft of claim 6, wherein, said first channel includes a first opening in the region of the shaft end facing an esophagus and a second opening in the region of the shaft end facing away from the esophagus.

8. The tube shaft of claim 5, wherein said second channel includes a ventilation opening for guiding respiratory air into an trachea.

9. The tube shaft of claim 8, wherein said second channel is provided with a connector for respiration.

10. The tube shaft of claim 9, wherein said connector distinguishes said first and second channels and prevents confusion.

11. The tube shaft of claim 9, wherein said connector is ISO standard 15 mm.

12. The tube shaft of claim 5, wherein said first and second cuffs are inflatable.
